(19) United States
(12) Patent Application Publication
(10) Pub. No.: US 2023/0107479 A1
(43) Pub. Date: Apr. 6, 2023

(54) METHODS AND COMPOSITIONS FOR PREVENTING AND TREATING FIBROSIS RESULTING FROM A CORONAVIRUS INFECTION

(71) Applicant: G3 Pharmaceuticals, Inc., Burlington, MA (US)

(72) Inventor: Pieter MUNTENDAM, Boxford, MA (US)

(21) Appl. No.: 17/913,545

(22) PCT Filed: Mar. 23, 2021
(86) PCT No.: PCT/US2021/023580

§ 371 Date: Sep. 22, 2022

Related U.S. Application Data
(60) Provisional application No. 62/993,179, filed on Mar. 23, 2020.

(51) Int. Cl. A61K 31/732; A61K 36/752; A61K 36/42; A61K 39/395; A61K 45/06; A61P 29/00; A61P 31/14; A61K 31/7004; A23L 33/00; A23L 33/125

(52) U.S. Cl. CPC A61K 31/732; A61K 36/752; A61K 36/42; A61K 39/3955; A61K 45/06; A61P 29/00; A61P 31/14; A61K 31/7004; A23L 33/40; A23L 33/125; A23V 2002/00

(57) ABSTRACT

The invention provides methods, compositions, and kits using a galectin-3 inhibitor to prevent and treat fibrosis resulting from a coronavirus infection, such as fibrosis in a subject's lung resulting from a coronavirus 2 infection.

### CROSS REFERENCE TO RELATED APPLICATION

This application is the national stage application of International (PCT) Patent Application Serial No. PCT/US2021/023580, filed Mar. 23, 2021, which claims the benefit of and priority to U.S. Provisional Application No. 62/993,179, filed Mar. 23, 2020, the contents of these applications are hereby incorporated by reference in their entirety.

### FIELD OF THE INVENTION

The invention provides methods, compositions, and kits using a galectin-3 inhibitor to prevent and treat fibrosis resulting from a coronavirus infection, such as fibrosis in a subject's lung resulting from a coronavirus 2 infection.

### BACKGROUND

Galectin-3 is a protein belonging to a specific sub-family of carbohydrate binding proteins (lectins) that recognize beta-galactosides. Galectins possess a carbohydrate recognition domain (CRD). The CRDs of various galectins differ in amino acid sequence outside of the conserved residues and this mediates specificity to different glycan ligands between galectins. Galectin-3 has both intracellular functions and extracellular functions and is actively secreted via a non-canonical pathway into the extracellular space and into the circulation. Binding of carbohydrates to the CRD results in modulation of galectin-3 activity in-vitro and in-vivo. Carbohydrate binding to the CRD and the resulting inhibition of galectin-3 is recognized as a potential therapeutic modality.

Galectin-3 has multiple biological functions. Galectin-3 has been identified as a mediator of fibrosis. Galectin-3-mediated fibrosis has been found to be an important underlying cause of a broad range of disease manifestations affecting the heart, lungs, kidney, vascular and liver system.

There remains an unmet need for methods of preventing and treating fibrosis resulting from a coronavirus infection. The present invention addresses this need and provides other related advantages.

### SUMMARY

The invention provides methods, compositions, and kits using a galectin-3 inhibitor to prevent and treat fibrosis resulting from a coronavirus infection, such as fibrosis in a subject's lung resulting from a severe acute respiratory syndrome coronavirus 2 infection. One benefit of the invention is that it provides a method for preventing and treating fibrosis in the subject's lungs, including fibrosis resulting from COVID-19, also known as Coronavirus disease 2019, due to an infection from the severe acute respiratory syndrome coronavirus 2 (SARS-CoV-2). The galectin-3 inhibitor may be, for example, a carbohydrate, such as a pectin.

One aspect of the invention provides a method of treating fibrosis resulting from a coronavirus infection, wherein the method comprises administering to a subject in need thereof a therapeutically effective amount of a galectin-3 inhibitor to treat the fibrosis. Another aspect of the invention provides a method of slowing the progression of fibrosis resulting from a coronavirus infection, wherein the method comprises administering to a subject in need thereof an effective amount of a galectin-3 inhibitor to slow the progression of the fibrosis. Another aspect of the invention provides a method of reducing the risk of fibrosis resulting from a coronavirus infection, wherein the method comprises administering to a subject in need thereof an effective amount of a galectin-3 inhibitor to reduce the risk of fibrosis in the subject. Yet another aspect of the invention provides a method of preventing the development of fibrotic tissue in a subject, wherein the method comprises administering to a subject in need thereof an effective amount of a galectin-3 inhibitor to prevent the development of fibrotic tissue in the subject, wherein the fibrotic tissue results from a coronavirus infection. Yet another aspect of the invention provides a method of treating or preventing a symptom of a coronavirus infection in a subject, wherein the method comprises administering to a subject in need thereof an effective amount of a galectin-3 inhibitor to treat or prevent a symptom of the coronavirus infection. Yet another aspect of the invention provides a method of reducing the impact of a pro-inflammatory cytokine in a patient suffering from a coronavirus infection, wherein the method comprises administering to a patient in need thereof an effective amount of a galectin-3 inhibitor, in order to reduce the impact of the pro-inflammatory cytokine.

Compositions for use in the methods are provided, along with medical kits containing materials and instructions for implementing the method.

### BRIEF DESCRIPTION OF THE DRAWINGS

The drawings show embodiments of the disclosed subject matter for the purpose of illustrating the invention. However, it should be understood that the present application is not limited to the precise arrangements and embodiments shown in the drawings.

FIG. 1 is a plot showing the quantification of ACE2 and TMPRSS2 in control and SARS-CoV-2 Spike protein-treated cells, represented as the mean±SEM of each group in arbitrary units (AU) normalized to the signal of stain-free protein gels or β-actin, as further described in Example 1.

FIGS. 2A and 2B are representative images of human cytokine array blots probed with the supernatant samples of controls (FIG. 2A) and SARS-CoV-2 Spike protein-treated cells (FIG. 2B) for 24 hours, as further described in Example 1.

FIG. 3 is a graph showing quantification of IL-6, CCL-2, IL-18, IL-27, INFγ, and PAI-1 secretion in HAECs treated with recombinant SARS-CoV-2 Spike protein, as further described in Example 1.

FIG. 4 is a graph showing IL-6 secretion in HAECs treated with recombinant SARS-CoV-2 Spike protein for 24, 48 and 72 hours, as further described in Example 1.

FIG. 5 is a graph showing CCL-2 secretion in HAECs treated with recombinant SARS-CoV-2 Spike protein for 24, 48 and 72 hours, as further described in Example 1.

FIG. 6 is a graph showing IL-18 secretion in HAECs treated with recombinant SARS-CoV-2 Spike protein for 24, 48 and 72 hours, as further described in Example 1.

FIG. 7 is a graph showing IL-27 secretion in HAECs treated with recombinant SARS-CoV-2 Spike protein for 24, 48 and 72 hours, as further described in Example 1.

FIG. 8 is a graph showing INFγ secretion in HAECs treated with recombinant SARS-CoV-2 Spike protein for 24, 48 and 72 hours, as further described in Example 1.

FIG. 9 is a graph showing PAI-1 secretion in HAECs treated with recombinant SARS-CoV-2 Spike protein for 24, 48 and 72 hours, as further described in Example 1.

FIG. 10 is a graph showing galectin-3 (Gal-3) secretion in HAECs treated with recombinant SARS-CoV-2 Spike protein for 24, 48 and 72 hours, as further described in Example 1.

FIG. 11 is a plot showing IL-6 secretion in HAECs pretreated with PP1 and then treated with recombinant SARS-CoV-2 Spike protein for 24, 48 and 72 hours, as further described in Example 1.

FIG. 12 is a plot showing CCL-2 secretion in HAECs pretreated with PP1 and then treated with recombinant SARS-CoV-2 Spike protein for 24, 48 and 72 hours, as further described in Example 1.

FIG. 13 is a plot showing IL-18 secretion in HAECs pretreated with PP1 and then treated with recombinant SARS-CoV-2 Spike protein for 24, 48 and 72 hours, as further described in Example 1.

FIG. 14 is a plot showing IL-27 secretion in HAECs pretreated with PP1 and then treated with recombinant SARS-CoV-2 Spike protein for 24, 48 and 72 hours, as further described in Example 1.

FIG. 15 is a plot showing INFγ secretion in HAECs pretreated with PP1 and then treated with recombinant SARS-CoV-2 Spike protein for 24 hours, as further described in Example 1.

FIG. 16 is a plot showing PAI-1 secretion in HAECs pretreated with PP1 and then treated with recombinant SARS-CoV-2 Spike protein for 24 hours, as further described in Example 1.

FIG. 17 is a plot showing IL-6 secretion in HAECs pretreated with recombinant SARS-CoV-2 Spike protein for 24 hours and then treated with PP1 for 48 or 72 hours, as further described in Example 1.

FIG. 18 is a plot showing CCL-2 secretion in HAECs pretreated with recombinant SARS-CoV-2 Spike protein for 24 hours and then treated with PP1 for 48 or 72 hours, as further described in Example 1.

FIG. 19 is a plot showing IL-18 secretion in HAECs pretreated with recombinant SARS-CoV-2 Spike protein for 24 hours and then treated with PP1 for 48 or 72 hours, as further described in Example 1.

FIG. 20 is a plot showing IL-27 secretion in HAECs pretreated with recombinant SARS-CoV-2 Spike protein for 24 hours and then treated with PP1 for 48 or 72 hours, as further described in Example 1.

### DETAILED DESCRIPTION OF THE INVENTION

The invention provides methods, compositions, and kits using a galectin-3 inhibitor to prevent and treat fibrosis resulting from a coronavirus infection, such as fibrosis in a subject's lung resulting from a severe acute respiratory syndrome coronavirus 2 infection. One benefit of the invention is that it provides a method for treating fibrosis in the subject's lungs, including fibrosis resulting from COVID-19, also known as Coronavirus disease 2019, due to an infection from the severe acute respiratory syndrome coronavirus 2 (SARS-CoV-2). The methods and compositions provide particular benefits to geriatric patients that may be more susceptible to a coronavirus infection. Various aspects of the invention are set forth below in sections; however, aspects of the invention described in one particular section are not to be limited to any particular section.

### Definitions

To facilitate an understanding of the present invention, a number of terms and phrases are defined below.

The terms “a,” “an” and “the” as used herein mean “one or more” and include the plural unless the context is inappropriate

As used herein, the term “subject” refers to organisms to be treated by the methods of the present invention. Such organisms preferably include, but are not limited to, mammals (e.g., murines, simians, equines, bovines, porcines, canines, felines, and the like), and most preferably includes humans.

As used herein, the term “effective amount” refers to the amount of a compound sufficient to effect beneficial or desired results. Unless specified otherwise, an effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, or disorder, or ameliorating a symptom thereof. As used herein, the term “preventing” refers to delaying or precluding onset of the condition, disease, or disorder.

As used herein, the term “pharmaceutical composition” refers to the combination of an active agent with a carrier, inert or active, making the composition especially suitable for therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Martin in Remington's Pharmaceutical Sciences, 15th Ed., Mack Publ. Co., Easton, Pa. [1975].

Compounds described herein may be formulated in the form of a pharmaceutically acceptable salt. As used herein, the term “pharmaceutically acceptable salt” refers to any pharmaceutically acceptable salt (e.g., acid or base) of a compound of the present invention which, upon administration to a subject, is capable of providing a compound of this invention. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₃, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate (mesylate), 2-naphthalenesulfonate, nicotinate, oxalate, palmoate, pectinate, persulfate, phenylpropionate, picrate, pivalate, propionate, succinate, sulfate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### I. Therapeutic Methods

The invention provides methods for treating fibrosis resulting from a coronavirus infection, slowing the progression of fibrosis resulting from a coronavirus infection, reducing the risk of fibrosis resulting from a coronavirus infection, and preventing the development of fibrotic tissue in a subject wherein the fibrotic tissue results from a coronavirus infection. The methods may be characterized according to, for example, the identity of the galectin-3 inhibitor, the dosing regimen, and preferred patient populations. Various aspects and embodiments of the therapeutic methods are described in the sections below. The sections are arranged for convenience and information in one section is not to be limited to that section, but may be applied to methods in other sections.

### A. First Method

One aspect of the invention provides a method of treating fibrosis resulting from a coronavirus infection. The method comprises administering to a subject in need thereof a therapeutically effective amount of a galectin-3 inhibitor to treat the fibrosis. The method may be further characterized according to additional exemplary features described below.

### B. Second Method

Another aspect of the invention provides a method of slowing the progression of fibrosis resulting from a coronavirus infection. The method comprises administering to a subject in need thereof an effective amount of a galectin-3 inhibitor to slow the progression of the fibrosis.

The method may be characterized according to, for example, the magnitude of reduction in the rate of progression of fibrosis resulting from a coronavirus infection. In certain embodiments, the method achieves at least a 25% reduction in the rate of progression of fibrosis compared to the average rate of progression of fibrosis in a subject having been infected by the coronavirus and not having received the galectin-3 inhibitor. In certain embodiments, the method achieves at least a 50% reduction in the rate of progression of fibrosis compared to the average rate of progression of fibrosis in a subject having been infected by the coronavirus and not having received the galectin-3 inhibitor. In certain embodiments, the method achieves at least a 90% reduction in the rate of progression of fibrosis compared to the average rate of progression of fibrosis in a subject having been infected by the coronavirus and not having received the galectin-3 inhibitor. The method may be further characterized according to additional exemplary features described below

### C. Third Method

Another aspect of the invention provides a method of reducing the risk of fibrosis resulting from a coronavirus infection. The method comprises administering to a subject in need thereof an effective amount of a galectin-3 inhibitor to reduce the risk of fibrosis in the subject.

The method may be further characterized according to, for example, the magnitude of the reduction in risk of fibrosis resulting from a coronavirus infection. In certain embodiments, the method produces at least a 5%, 10%, 20%, 30%, 40%, 50%, 60%, 70%, 80%, 90%, or 95% reduction in the risk of fibrosis. In certain embodiments, the method achieves at least a 25% reduction in risk of fibrosis. In certain embodiments, the method achieves at least a 50% reduction in risk of fibrosis. In certain embodiments, the method achieves at least a 90% reduction in risk of fibrosis.

The method may be further characterized according to additional exemplary features described below.

### D. Fourth Method

Another aspect of the invention provides a method of preventing the development of fibrotic tissue in a subject. The method comprises administering to a subject in need thereof an effective amount of a galectin-3 inhibitor to prevent the development of fibrotic tissue in the subject, wherein the fibrotic tissue results from a coronavirus infection.

Yet another aspect of the invention provides a method of preventing the development of fibrotic tissue in a subject. The method comprises administering to a subject in need thereof an effective amount of a galectin-3 inhibitor to prevent the growth of fibrotic tissue in the subject, wherein the fibrotic tissue results from a coronavirus infection.

The methods may be characterized according to, for example, the location of the fibrotic tissue. For example, in certain embodiments, the fibrotic tissue is located in the lung of the subject. In certain embodiments, the fibrotic tissue is pulmonary fibrotic tissue resulting from a coronavirus infection. In certain embodiments, the fibrotic tissue is located in the heart of the subject. In certain embodiments, the fibrotic tissue is located in the kidney of the subject.

The method may be further characterized according to additional exemplary features described below.

### E. Fifth Method

Another aspect of the invention provides a method of treating or preventing a symptom of a coronavirus infection in a subject. The method comprises administering to a subject in need thereof an effective amount of a galectin-3 inhibitor to treat or prevent a symptom of the coronavirus infection. In certain embodiments, the method treats or prevents a symptom of COVID-19.

In certain embodiments, the method treats a symptom of a coronavirus infection in a subject. In certain embodiments, the method prevents a symptom of a coronavirus infection in a subject.

In certain embodiments, a symptom of the coronavirus infection is one or more of fever, chills, cough, shortness of breath, difficulty breathing, fatigue, muscle aches, body aches, headache, loss of taste or smell, sore throat, congestion, runny nose, nausea, vomiting, or diarrhea. In certain embodiments, a symptom of the coronavirus infection is one or more of fever, cough, shortness of breath, difficulty breathing, fatigue, congestion, or runny nose.

The method may be further characterized according to additional exemplary features described below.

### F. Sixth Method

Another aspect of the invention provides a method of reducing the impact of a pro-inflammatory cytokine in a patient suffering from a coronavirus infection. The method comprises administering to a subject in need thereof an effective amount of a galectin-3 inhibitor, in order to reduce the impact of the pro-inflammatory cytokine. In certain embodiments, the pro-inflammatory cytokine is IL-1, IL-2, IL-6, or IL-7. In certain embodiments, the pro-inflammatory cytokine is IL-1. In certain embodiments, the pro-inflammatory cytokine is IL-2. In certain embodiments, the pro-inflammatory cytokine is IL-6. In certain embodiments, the pro-inflammatory cytokine is IL-7.

The method may be further characterized according to, for example, the magnitude of the reduction of the impact of the pro-inflammatory cytokine resulting from a coronavirus infection. In certain embodiments, the method produces at least a 5%, 10%, 20%, 30%, 40%, 50%, 60%, 70%, 80%, 90%, or 95% reduction in the impact of the pro-inflammatory cytokine. In certain embodiments, the method achieves at least a 25% reduction in the impact of the pro-inflammatory cytokine. In certain embodiments, the method achieves at least a 50% reduction in the impact of the pro-inflammatory cytokine. In certain embodiments, the method achieves at least a 90% reduction in the impact of the pro-inflammatory cytokine.

The method may be further characterized according to additional exemplary features described below.

### G. Additional Exemplary Features of the First, Second, Third, Fourth, Fifth and Sixth Therapeutic Methods

The First, Second, Third, Fourth, Fifth and Sixth Therapeutic Methods described herein may be further characterized according to, for example, the identity of the galectin-3 inhibitor, the dosing regimen, the type and cause of the fibrosis, preferred patient populations, and other features described herein below. A more thorough description of such features is provided below. The invention embraces all permutations and combinations of these features, where appropriate.

1. Galectin-3 Inhibitors

The method may be characterized according to the identity of the galectin-3 inhibitor. For example, in certain embodiments, the galectin-3 inhibitor is a carbohydrate, protein, lipid, nucleic acid, or small organic compound. In certain embodiments, the galectin-3 inhibitor comprises a carbohydrate. In certain embodiments, the galectin-3 inhibitor comprises a polysaccharide. In certain embodiments, the galectin-3 inhibitor is a carbohydrate. In certain embodiments, the galectin-3 inhibitor is a polysaccharide.

In certain embodiments, the galectin-3 inhibitor comprises a pectin. In certain embodiments, the galectin-3 inhibitor is a pectin. Pectins are polysaccharides derived from plant cell walls, especially from apple and citrus fruits. A pectin used may be a full-length pectin or may be a pectin fragment. In certain embodiments, the pectin fragment may be purified according to procedures described in the literature. The pectin may be characterized according to its molecular weight. In certain embodiments, the pectin has a molecular weight in the range of from about 50 kDa to about 150 kDa, from about 60 kDa to about 130 kDa, from about 50 kDa to about 100 kDa, from about 30 kDa to about 60 kDa, from about 10 kDa to about 50 kDa, from about 10 kDa to about 30 kDa, from about 5 kDa to about 20 kDa, or from about 1 kDa to about 10 kDa.

In certain embodiments, the polysaccharide is a pumpkin pectin. In certain embodiments, the polysaccharide is an apple pectin. In certain embodiments, the polysaccharide is a citrus fruit pectin. In certain embodiments, the polysaccharide is a sugar beet pectin. In certain embodiments, the polysaccharide is a pear pectin. In certain embodiments, the polysaccharide is a potato pectin. In certain embodiments, the polysaccharide is a carrot pectin.

In certain embodiments, the galectin-3 inhibitor comprises a polysaccharide isolated from a plant material. In certain embodiments, the plant material is a member of the genus Cucurbita. In certain embodiments, the polysaccharide is isolated from C. moschata, C. argyrosperma, C. fwifolia, C. maxima, or C. pepo.

In certain embodiments, the polysaccharide comprises galactose. In certain embodiments, the polysaccharide comprises a rhamnogalacturonan I (RG-I) domain. In certain embodiments, the RG-I domain comprises β-D-galactan, α-L-arabinofuranosyl, or combinations thereof. In certain embodiments, the polysaccharide comprises a homogalacturonan (HG) domain.

In certain embodiments, the polysaccharide has a molecular weight of about 5 kDa to about 70 kDa. In certain embodiments, the polysaccharide has a molecular weight of about 20 kDa to about 30 kDa. In certain embodiments, the polysaccharide has a molecular weight of about 20 kDa to about 25 kDa. In certain embodiments, the polysaccharide has a molecular weight of about 5 kDa to about 25 kDa. In certain embodiments, the polysaccharide has a molecular weight of about 17 kDa to about 23 kDa. In certain embodiments, the molecular weight of the polysaccharide is about 17.5 kDa. In certain embodiments, the galectin-3 inhibitor comprises or is a polysaccharide described in PCT Application Publication WO 2019/143924A1, the entirety of which is incorporated by reference herein.

In certain embodiments, the galectin-3 inhibitor comprises Modified Citrus Pectin (MCP). In certain embodiments, the galectin-3 inhibitor is MCP. MCP is different from other pectins, as it is modified from organic citrus pectin to reduce the molecular weight of the pectin molecule, such as to between about 10 kDa and about 30 kDa or between about 5 kDa and about 20 kDa.

In certain embodiments, the galectin-3 inhibitor is a pectic compound. Pectic compounds are derived from pectins, where a substantial portion of the pectin backbone has been removed.

In certain embodiments, the galectin-3 inhibitor comprises an artificial polysaccharide. In certain embodiments, the galectin-3 inhibitor is an artificial polysaccharide. In certain embodiments, the artificial polysaccharide is selected from GR-MD-02 and GM-CT-01 (Davanat™)

In certain embodiments, the polysaccharide is modified with one or more non-naturally occurring chemical moieties. In certain embodiments, the polysaccharide is given one or more modifications concurrent with or subsequent to isolation from a plant material. In certain embodiments, the one or more modifications include alkylation, amidation, quaternization, thiolation, sulfation, oxidation, chain elongation, e.g., cross-linking, grafting, etc., depolymerization by chemical, physical, or biological processes including enzymatic process, etc., or combinations thereof.

In certain embodiments, the galectin-3 inhibitor comprises a chemically modified polysaccharide. In certain embodiments, the galectin-3 inhibitor is a chemically modified polysaccharide. In certain embodiments, the chemically modified polysaccharide is TD139.

In certain embodiments, the polysaccharide has a galectin-3 binding affinity greater than that of potato galactan. In certain embodiments, the polysaccharide inhibits galectin-3 activity at concentrations of the polysaccharide below 2 mM. In certain embodiments, the polysaccharide inhibits galectin-3 activity at concentrations of the polysaccharide at about 1.26 mM.

In certain embodiments, the galectin-3 inhibitor comprises a protein, antibody, galectin binding protein (GBP) interaction fusion protein, peptide aptamer, Avimer, Fab, sFv, Adnectin, ligand, nucleic acid, or lipid. In certain embodiments, the galectin-3 inhibitor comprises an antibody, galectin binding protein (GBP) interaction fusion protein, peptide aptamer, Avimer, Fab, sFv, Adnectin, ligand, or nucleic acid.

In certain embodiments, the galectin-3 inhibitor comprises a protein. In certain embodiments, the galectin-3 inhibitor comprises an antibody, galectin binding protein (GBP) interaction fusion protein, peptide aptamer, Avimer, Fab, sFv, Adnectin, or ligand.

In certain embodiments, the galectin-3 inhibitor comprises an antibody. In certain embodiments, the galectin-3 inhibitor comprises a primary, secondary, monoclonal, polyclonal, human, humanized, or chimeric antibody. In certain embodiments, the galectin-3 inhibitor comprises a primary antibody. In certain embodiments, the galectin-3 inhibitor comprises a secondary antibody. In certain embodiments, the galectin-3 inhibitor comprises a monoclonal or polyclonal antibody. In certain embodiments, the galectin-3 inhibitor comprises a monoclonal antibody. In certain embodiments, the galectin-3 inhibitor comprises a polyclonal antibody. In certain embodiments, the galectin-3 inhibitor comprises a human antibody. In certain embodiments, the galectin-3 inhibitor comprises a humanized antibody. In certain embodiments, the galectin-3 inhibitor comprises chimeric antibody.

In certain embodiments, the galectin-3 inhibitor comprises antibody 87B5. In certain embodiments, the galectin-3 inhibitor is antibody 87B5. In certain embodiments, the galectin-3 inhibitor comprises antibody M3/38. In certain embodiments, the galectin-3 inhibitor is antibody M3/38.

In certain embodiments, the galectin-3 inhibitor comprises an antibody fragment. In certain embodiments, the galectin-3 inhibitor comprises a single chain Fv antibody (sFv). In certain embodiments, the galectin-3 inhibitor comprises an antigen-binding fragment (Fab).

In certain embodiments, the galectin-3 inhibitor comprises a galectin binding protein (GBP) interaction fusion protein. In certain embodiments, the galectin-3 inhibitor comprises a peptide aptamer. In certain embodiments, the galectin-3 inhibitor comprises an Avimer. In certain embodiments, the galectin-3 inhibitor comprises an Adnectin. In certain embodiments, the galectin-3 inhibitor comprises an AFFIBODY® ligand.

In certain embodiments, the galectin-3 inhibitor comprises a nucleic acid. In certain embodiments, the galectin-3 inhibitor comprises DNA. In certain embodiments, the galectin-3 inhibitor comprises RNA. In certain embodiments, the galectin-3 inhibitor comprises a nucleotide aptamer.

In certain embodiments, the galectin-3 inhibitor comprises a lipid. In certain embodiments, the galectin-3 inhibitor comprises a membrane lipid.

In certain embodiments, the galectin-3 inhibitor is a protein, nucleic acid, or lipid.

In certain embodiments, the galectin-3 inhibitor is a protein, antibody, galectin binding protein (GBP) interaction fusion protein, peptide aptamer, Avimer, Fab, sFv, Adnectin, ligand, nucleic acid, or lipid. In certain embodiments, the galectin-3 inhibitor is an antibody, galectin binding protein (GBP) interaction fusion protein, peptide aptamer, Avimer, Fab, sFv, Adnectin, ligand, or nucleic acid. In certain embodiments, the galectin-3 inhibitor is an antibody. In certain embodiments, the galectin-3 inhibitor comprises an antibody.

In certain embodiments, the galectin-3 inhibitor is a protein. In certain embodiments, the galectin-3 inhibitor is an antibody, galectin binding protein (GBP) interaction fusion protein, peptide aptamer, Avimer, Fab, sFv, Adnectin, or ligand.

In certain embodiments, the galectin-3 inhibitor is an antibody. In certain embodiments, the galectin-3 inhibitor is a primary, secondary, monoclonal, polyclonal, human, humanized, or chimeric antibody. In certain embodiments, the galectin-3 inhibitor is a primary antibody. In certain embodiments, the galectin-3 inhibitor is a secondary antibody. In certain embodiments, the galectin-3 inhibitor is a monoclonal or polyclonal antibody. In certain embodiments, the galectin-3 inhibitor is a monoclonal antibody. In certain embodiments, the galectin-3 inhibitor is a polyclonal antibody. In certain embodiments, the galectin-3 inhibitor is a human antibody. In certain embodiments, the galectin-3 inhibitor is a humanized antibody. In certain embodiments, the galectin-3 inhibitor is chimeric antibody.

In certain embodiments, the galectin-3 inhibitor is an antibody fragment. In certain embodiments, the galectin-3 inhibitor is a single chain Fv antibody (sFv). In certain embodiments, the galectin-3 inhibitor is an antigen-binding fragment (Fab).

In certain embodiments, the galectin-3 inhibitor is a galectin binding protein (GBP) interaction fusion protein. In certain embodiments, the galectin-3 inhibitor is a peptide aptamer. In certain embodiments, the galectin-3 inhibitor is an Avimer. In certain embodiments, the galectin-3 inhibitor is an Adnectin. In certain embodiments, the galectin-3 inhibitor is an AFFIBODY® ligand.

In certain embodiments, the galectin-3 inhibitor is a nucleic acid. In certain embodiments, the galectin-3 inhibitor is DNA. In certain embodiments, the galectin-3 inhibitor is RNA. In certain embodiments, the galectin-3 inhibitor is a nucleotide aptamer.

In certain embodiments, the galectin-3 inhibitor is a lipid. In certain embodiments, the galectin-3 inhibitor is a membrane lipid.

In certain embodiments, the galectin-3 inhibitor is a small organic molecule.

In certain embodiments, the galectin-3 inhibitor is a component in food, such as a vegetable (e.g., squash and pumpkin), fruit (e.g., pear, apple, guavas, quince, plum, gooseberry, and oranges), or other food product that contains pectin. In certain embodiments, the galectin-3 inhibitor is a component in a food product, such as a vegetable (e.g., squash and pumpkin), fruit (e.g., pear, apple, guavas, quince, plum, gooseberry, and oranges), or other food product that contains pectin. In certain embodiments, the method of administering a galectin-3 inhibitor comprises administering food. In certain embodiments, the method of administering a galectin-3 inhibitor comprises administering food containing therapeutic amounts of pectin.

In certain embodiments, the galectin-3 inhibitor is a component in nutritional product. In certain embodiments, the galectin-3 inhibitor is formulated as a component of a nutritional product. The nutritional product may contain, for example, components from a vegetable (e.g., squash and pumpkin), fruit (e.g., pear, apple, guavas, quince, plum, gooseberry, and oranges), or other plant that contains pectin. In certain embodiments, the nutritional product contains elevated levels of pectin relative to the amount of pectin in source materials used to prepare the nutritional product. In certain embodiments, the method of administering a galectin-3 inhibitor comprises administering a nutritional supplement.

2. Dosing Regimen

In certain embodiments, the method may be characterized based on the amount of galectin-3 inhibitor being administered and/or frequency with which the galectin-3 inhibitor is administered to the subject. The galectin-3 inhibitor can be dosed, for example, based on the weight of the subject or as a fixed dose. In certain embodiments, the galectin-3 inhibitor is administered 1, 2, or 3 times per day. In certain embodiments, each administration of galectin-3 inhibitor provides from about 0.1 g to about 0.5 g, from about 0.5 to about 1.0, from about 1.0 to about 2.0 g, or from about 2 to about 3 g of galectin-3 inhibitor. In certain embodiments, a dose of modified citrus pectin is from about 1 g to about 10 g, from about 3 g to about 7 g, or about 5 g.

In certain embodiments, the galectin-3 inhibitor is administered enterally or parenterally, e.g., oral, sublingual, rectal, intravenous, subcutaneous, topical, transdermal, intradermal, transmucosal, intraperitoneal, intramuscular, intracapsular, intraorbital, intracardiac, transtracheal, subcutaneous, subcuticular, intraarticular, subcapsular, subarachnoid, intraspinal, epidural and intrasternal injection, infusion, etc., or combinations thereof.

In certain embodiments, the galectin-3 inhibitor is administered orally to the subject.

In certain embodiments, the galectin-3 inhibitor is administered to the subject after the subject has stopped exhibiting any fever, cough, difficulty breathing, fatigue, or digestive system distress due to the coronavirus infection. Administration of the galectin-3 inhibitor after the subject has stopped exhibiting any fever, cough, difficulty breathing, fatigue, or digestive system distress due to the coronavirus infection may minimize the risk of complications and/or adverse side effects.

3. Etiology of Fibrosis

The methods may be characterized based on the location and/or etiology of the fibrosis. In certain embodiments, the fibrosis comprises fibrosis in the subject's lung. In certain embodiments, the fibrosis comprises pulmonary fibrosis resulting from a coronavirus infection. In certain embodiments, the fibrosis comprises fibrosis in the subject's heart. In certain embodiments, the fibrosis comprises fibrosis in the subject's kidney.

In certain embodiments, the fibrosis is fibrosis in the subject's lung. In certain embodiments, the fibrosis is pulmonary fibrosis resulting from a coronavirus infection. In certain embodiments, the fibrosis is fibrosis in the subject's heart. In certain embodiments, the fibrosis is fibrosis in the subject's kidney.

In certain embodiments, the coronavirus is selected from severe acute respiratory syndrome coronavirus I (SARS-CoV), Middle East respiratory syndrome coronavirus (MERS coronavirus), severe acute respiratory syndrome virus II (SARS-CoV-2), or Bat SARS-like coronavirus WIV1. In certain embodiments, the fibrosis develops in the subject as a result of a disease resulting from a coronavirus infection. For example, in certain embodiments, the fibrosis develops as a result of coronavirus disease 2019 (COVID-19), after infection with SARS-CoV-2.

In certain embodiments, the coronavirus infection is an infection by a severe acute respiratory syndrome—related coronavirus (SARSr-CoV). In certain embodiments, the coronavirus infection is an infection by a Sarbecovirus (beta-CoV lineage B). In certain embodiments, the coronavirus infection is an infection by SARS-CoV-2.

In certain embodiments, the coronavirus infection is an infection by a variant of SARS-CoV-2. In certain embodiments, the coronavirus infection is an infection by a variant of SARS-CoV-2 having the spike protein of SARS-CoV-2. In certain embodiments, the coronavirus infection is an infection by SARS-CoV-2 or a variant thereof selected from B.1.351, Cluster 5, Lineage B.1.1.207, Lineage B.1.1.7, Variant of Concern 202102/02, Lineage B.1.1.317, Lineage B.1.1.318, Lineage B.1.351, Lineage B.1.429, Lineage B.1.525, Lineage P.1, D614G, E484K, N501Y, S477G/N, and P681H.

In certain embodiments, the coronavirus infection is an infection by SARS-CoV-2 or variant thereof having a mutation at 1, 2, 3, 4, or 5 amino acids. In certain embodiments, the coronavirus infection is an infection by SARS-CoV-2 or variant thereof having a mutation at up to 10 amino acids. In certain embodiments, the coronavirus infection is an infection by SARS-CoV-2 or variant thereof having a mutation at up to 25 amino acids.

4. Patient Populations that May Derive Particular Benefits from the Therapeutic Methods

The method may be further characterized according to the subject suffering from fibrosis resulting from coronavirus infection. For example, in certain embodiments, the subject is a human. In certain embodiments, the subject is an adult human. In certain embodiments, the subject is a geriatric human.

In certain embodiments, the subject has a concentration of galectin-3 in a bodily fluid that is greater than the average concentration of galectin-3 in the same bodily fluid of a healthy subject. In certain embodiments, the bodily fluid is blood plasma. In certain embodiments, the bodily fluid is blood serum. In certain embodiments, the concentration of galectin-3 in a bodily fluid of the subject is at least 5%, 10%, 15%, 20%, 25%, 30%, 35%, 40%, 45%, 50%, 55%, 60%, 70%, 75%, 80%, 85%, 90%, 95%, or 100% greater than the average concentration of galectin-3 in the same bodily fluid of a healthy subject.

In certain embodiments, the subject features a concentration of galectin-3 in a bodily fluid that increases over time. To illustrate, in certain embodiments the subject has a concentration of galectin-3 in a bodily fluid that is greater than the concentration of galectin-3 in the same type of bodily fluid observed in the subject 1, 2, 3, 4, 5, 6, 7, 10, 12, or 14 days prior. In certain embodiments, the bodily fluid is blood plasma. In certain embodiments, the bodily fluid is blood serum. In certain embodiments, the concentration of galectin-3 in a bodily fluid of the subject is at least 5%, 10%, 15%, 20%, 25%, 30%, 35%, 40%, 45%, 50%, 55%, 60%, 70%, 75%, 80%, 85%, 90%, 95%, or 100% greater than the concentration of galectin-3 in the same type of bodily fluid observed in the subject 1, 2, 3, 4, 5, 6, 7, 10, 12, or 14 days prior.

In certain embodiments, the subject suffers from pulmonary fibrosis and exhibits one or more symptoms typical of pulmonary fibrosis. In certain embodiments, the subject exhibits one or more symptoms of pulmonary fibrosis including, but not limited to, shortness of breath (dyspnea), cough, fatigue, unexplained weight loss, joint pain, muscle pain, clubbing of tips of their fingers and/or toes, blood clots, and lung collapse.

5. Therapeutic Improvements & Other Characteristics

The method may be further characterized according to the therapeutic benefit of administration of the galectin-3 inhibitor to the subject. For example, in certain embodiments, the method produces at least a 5%, 10%, 20%, 30%, 40%, 50%, 60%, 70%, 80%, 90%, or 95% reduction in a symptom of the fibrosis. In certain embodiments, the method achieves at least a 25% reduction in a symptom of fibrosis. In certain embodiments, the method achieves at least a 50% reduction in a symptom of fibrosis. In certain embodiments, the method achieves at least a 90% reduction in a symptom of fibrosis. In certain embodiments, the symptom of fibrosis is the volume of fibrotic tissue. In certain embodiments, the symptom of fibrosis is the number of fibrotic scars.

In certain embodiments, the method reduces the severity of one or more symptoms of pulmonary fibrosis in the subject, including, but not limited to, shortness of breath, cough, fatigue, unexplained weight loss, joint pain, muscle pain, clubbing of tips of their fingers and/or toes, blood clots, and lung collapse. In certain embodiments, the method produces at least a 5%, 10%, 20%, 30%, 40%, 50%, 60%, 70%, 80%, 90%, or 95% reduction in the severity or frequency of one or more symptoms of pulmonary fibrosis in the subject.

6. Combination Therapy

Another aspect of the invention provides for combination therapy. Galectin-3 inhibitors described herein may be used in combination with additional therapeutic agents to treat medical disorders, such as a fibrosis.

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

One or more other therapeutic agents may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition of the invention are administered as a multiple dosage regimen more than 24 hours apart.

As used herein, the term “combination,” “combined,” and related terms refers to the simultaneous or sequential administration of therapeutic agents in accordance with this invention. For example, a compound of the present invention can be administered with one or more other therapeutic agent(s) simultaneously or sequentially in separate unit dosage forms or together in a single unit dosage form. Accordingly, the present invention provides a single unit dosage form comprising a compound of the current invention, one or more other therapeutic agent(s), and a pharmaceutically acceptable carrier, adjuvant, or vehicle.

The amount of a compound of the invention and one or more other therapeutic agent(s) (in those compositions which comprise an additional therapeutic agent, such as a second anti-cancer agent, as described above) that can be combined with the carrier materials to produce a single dosage form varies depending upon the host treated and the particular mode of administration. Preferably, a composition of the invention should be formulated so that a dosage of between 0.01-100 mg/kg body weight/day of a compound of the invention can be administered.

In those compositions which comprise one or more other therapeutic agent(s), the one or more other therapeutic agent(s) and a compound of the invention can act synergistically. Therefore, the amount of the one or more other therapeutic agent(s) in such compositions may be less than that required in a monotherapy utilizing only that therapeutic agent. In such compositions a dosage of between 0.01-1,000 g/kg body weight/day of the one or more other therapeutic agent(s) can be administered.

The amount of one or more other therapeutic agent(s) present in the compositions of this invention is preferably no more than the amount that would normally be administered in a composition comprising that therapeutic agent as the only active agent. Preferably the amount of one or more other therapeutic agent(s) in the presently disclosed compositions ranges from about 50% to 100% of the amount normally present in a composition comprising that agent as the only therapeutically active agent. In some embodiments, one or more other therapeutic agent(s) is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA-approved therapeutic agent is approved for dosing per the FDA label insert.

In certain embodiments, the additional therapeutic agent is a mineralocorticoid receptor antagonist, angiotensin-converting enzyme (ACE) inhibitor, angiotensin-receptor blocker (ARB), anti-inflammatory agent, or combination thereof. In certain embodiments, the additional therapeutic agent is a mineralocorticoid receptor antagonist, angiotensin-converting enzyme (ACE) inhibitor, angiotensin-receptor blocker (ARB), non-steroidal anti-inflammatory drug (NSAID), inhibitor of IL-6, or combination thereof.

In certain embodiments, the additional therapeutic agent is a mineralocorticoid receptor antagonist. In certain embodiments, the additional therapeutic agent is a mineralocorticoid receptor antagonist selected from spironolactone, eplerenone, canrenone, finerenone, or mexrenone.

In certain embodiments, the additional therapeutic agent is an angiotensin-converting enzyme inhibitor. In certain embodiments, the additional therapeutic agent is an angiotensin-converting enzyme inhibitor selected from alacepril, captopril, zefnopril, enalapril, ramipril, quinapril, perindopril, lisinopril, benazepril, imidapril, trandolapril, cilazapril, fosinopril, or arfalasin. In certain embodiments, the additional therapeutic agent is an angiotensin-converting enzyme inhibiting peptide disclosed in Kumar, et al., Nucleic Acids Research, 2015, 43, 956-962, which is hereby incorporated by reference.

In certain embodiments, the additional therapeutic agent is an angiotensin-receptor blocker. In certain embodiments, the additional therapeutic agent is an angiotensin-receptor blocker selected from azilsartan, candesartan, eprosartan, irbesartan, losartan, olmesartan, telmisartan, or valsartan.

In certain embodiments, the additional therapeutic agent is a non-steroidal anti-inflammatory drug. In certain embodiments, the additional therapeutic agent is a non-steroidal anti-inflammatory drug selected from ibuprofen, naproxen, diclofenac, celecoxib, mefenamic acid, etoricoxib, indomethacin, high-dose aspirin, ketoprofen, ketorolac, piroxicam, salsalate, tolmetin, sulindac, oxaprozin, etodolac, or nabumetone.

In certain embodiments, the additional therapeutic agent is an inhibitor of IL-6. In certain embodiments, the additional therapeutic agent is an inhibitor of IL-6 selected from sarilumab, tocilizumab, siltuximab, olokizumab, elsilimomab, clazakizumab, sirukumab, or levilimab.

### II. Compositions for Medical Use

Galectin-3 inhibitors described herein may be used to prevent and treat fibrosis resulting from a coronavirus infection, as described above. The use may be according to a method described herein. For example, one aspect of the invention provides a galectin-3 inhibitor for use in treating fibrosis resulting from a coronavirus infection. Another aspect of the invention provides a galectin-3 inhibitor for use in slowing the progression of fibrosis resulting from a coronavirus infection. Another aspect of the invention provides a galectin-3 inhibitor for use in reducing the risk of fibrosis resulting from a coronavirus infection. Another aspect of the invention provides a galectin-3 inhibitor for use in preventing the development of fibrotic tissue resulting from a coronavirus infection.

Embodiments described herein in connection with the methods for treatment may be applied in connection with the galectin-3 inhibitors for use.

### III. Preparation of a Medicament

Galectin-3 inhibitors described herein may be used in the preparation of a medicament to prevent and treat fibrosis resulting from a coronavirus infection, as described above. For example, one aspect of the invention provides for the use of a galectin-3 inhibitor described herein in the preparation of a medicament for treating fibrosis resulting from a coronavirus infection. Another aspect of the invention provides for the use of a galectin-3 inhibitor described herein in the preparation of a medicament for slowing the progression of fibrosis resulting from a coronavirus infection. Another aspect of the invention provides for the use of a galectin-3 inhibitor described herein in the preparation of a medicament for reducing the risk of fibrosis resulting from a coronavirus infection. Another aspect of the invention provides for the use of a galectin-3 inhibitor described herein in the preparation of a medicament for preventing the development of fibrotic tissue resulting from a coronavirus infection.

Embodiments described herein in connection with the methods for treatment may be applied in connection with the galectin-3 inhibitors for use in the preparation of a medicament.

### IV. Pharmaceutical Compositions

As indicated above, the invention provides pharmaceutical compositions, which comprise a compound described above and a pharmaceutically acceptable carrier. The pharmaceutically acceptable carrier may contain additive(s) and/or diluent(s). The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “therapeutically effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising a compound described herein in a therapeutically effective amount for the treatment of a medical disorder described herein.

### V. Kits

Another aspect of the invention provides a medical kit comprising, for example, (i) a galectin-3 inhibitor, and (ii) instructions for use according to a method described herein (e.g., treating fibrosis resulting from a coronavirus infection according to a method described herein).

### EXAMPLES

The invention now being generally described, will be more readily understood by reference to the following examples, which are included merely for purposes of illustration of certain aspects and embodiments of the present invention, and are not intended to limit the invention.

### Example 1—Analysis of Ability of Pumpkin Pectin to Prevent and Reverse Secretion of Inflammatory Molecules and a Thrombosis Marker by Human Vascular Endothelial Cells Exposed to Sars-Cov2 Spike (S) Protein

Human vascular endothelial cells (HAECs) were treated with recombinant SARS-COV2 Spike (S) protein resulting in enhanced secretion of inflammatory molecules (interleukin-6, monocyte chemoattractant protein-1, interleukin-18, interleukin-27 and interferon-γ) as well as in the thrombosis marker plasminogen activator inhibitor (PAI)-1. This was prevented and reversed by pumpkin pectin (PP1), which is galectin-3 inhibitor. The pumpkin pectin (PP1) was obtained using a process that entails treating pumpkin residue with an enzyme under aqueous conditions to produce a mixture, and then isolating pumpkin pectin (PP1) from the mixture.

Experimental procedures and results are described below.

### Cell Culture and Treatments

Human aortic endothelial cells (HAECs) from 4 different male donors were obtained from Promocell. HAECs were grown in endothelial cell growth media following the manufacturer's instructions (Promocell). All assays were carried out at 37° C., 95% sterile air and 5% CO2 in a saturation humidified incubator. Cells were used between passages 3-5.

HAECs were treated with recombinant SARS-CoV2 Spike protein (1 μg/ml, R&D Systems) for 24 to 72 hours for protein studies. Cells were treated with PP1 (1 mg/mL) 1 hour before Spike stimulation to assess the preventive effect of the aforementioned the Gal-3 inhibitor. Cell supernatants were collected at 24, 48 and 72 hours. In another set of experiments, HAECs were treated with recombinant Spike (1 μg/ml) for 24 hours and then co-treated with PP1 (1 mg/ml) for a further 24 and 48 hours to study if Galectin-3 antagonism can revert the pathological effect of SARS-CoV2 Spike protein on HAECs.

### Western Blot Analysis

Aliquots of 10 μg of total proteins were prepared from cells, electrophoresed on SDS polyacrylamide gels and transferred to Hybond-c Extra nitrocellulose membranes (BIO-RAD). Membranes were incubated with primary antibodies for: ACE2 (1:50, ABCAM), TMPRSS2 (1:100, ABCAM), phosphorylated NFκB (1:200, SANTA CRUZ BIOTECHNOLOGY), total NFκB (1:200, SANTA CRUZ BIOTECHNOLOGY) or β-actin (1:1000, SIGMA ALDRICH). Stain free detection was also used as loading control. After washing, detection was made through incubation with peroxidase-conjugated secondary antibody, and developed using an ECL chemiluminescence kit (Amersham). After densitometric analyses, optical density values were expressed as arbitrary units. All Western Blots were performed at least in triplicate for each experimental condition.

### Cytokine Array

After incubation time as indicated (24 hours), cell supernatants were collected and analyzed using a human cytokine array kit following manufacturer's instructions (R&D Systems). A pool of at least 12 supernatants per condition of 3 independent experiments were used. The results were normalized to the control condition in each cell type. Data were expressed as a fold change relative to controls.

### ELISA

Secretion of IL-6, CCL-2, IL-18, IL-21, IL-27, INFγ, PAI-1 and Gal-3 was assessed in cell supernatants by ELISA according to the manufacturer's instructions (R&D SYSTEMS).

### Statistical Analyses

Continuous variables are shown as mean±SEM. Normal distribution was verified by means of the Kolmogorov-Smirnov test. Normally distributed data was compared using a one-way analysis of variance (ANOVA) test followed by a Dunnett's multiple comparison post hoc analysis. Non-parametric data was analyzed using the Kruskal Wallis test followed by Mann Whitney U test. Statistical significance was accepted at p<0.05. Analyses were performed using GraphPad Prism® 5.0 (GRAPHPAD SOFTWARE INC).

### SARS-CoV-2 Spike Protein Effects on HAECs

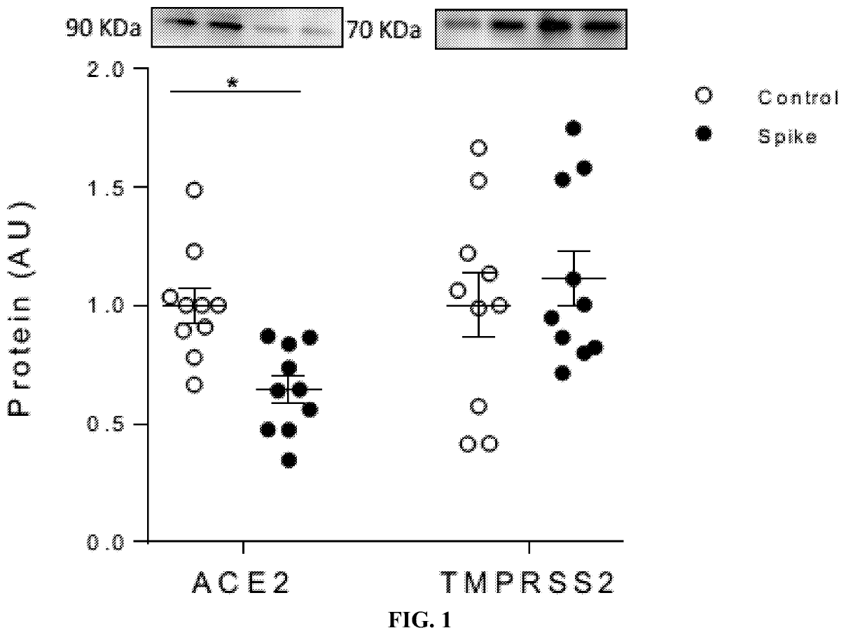

The expression of ACE2 and TMPRSS2 was confirmed in HAECs (FIG. 1; the graph represents the mean±SEM of each group in arbitrary units (AU) normalized to the signal of stain-free protein gels or β-actin. n=10 wells per condition from 4 independent HAECs donors. *p<0.05 vs. control). Stimulation with SARS-CoV-2 Spike protein for 24 hours decreased (36%, p=0.0012) ACE2 expression and did not modify TMPRSS2 expression (FIG. 1).

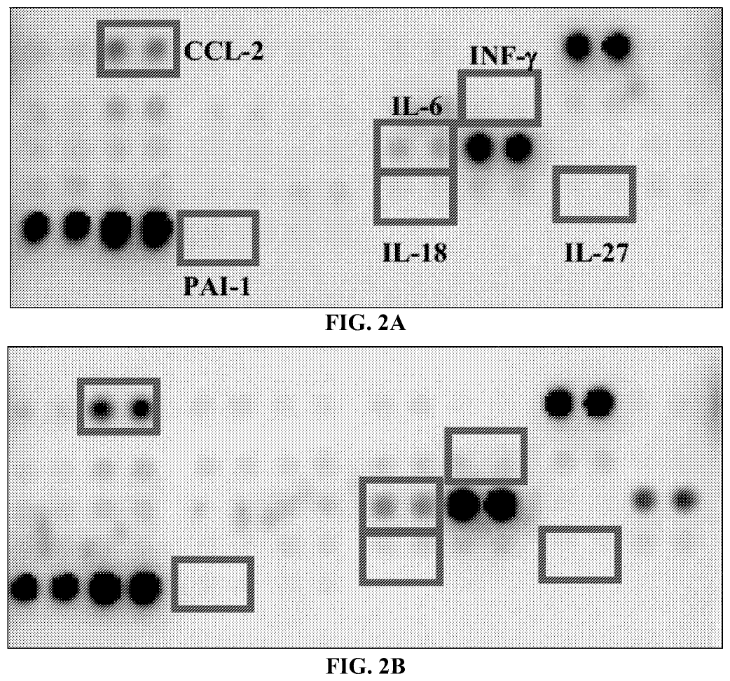

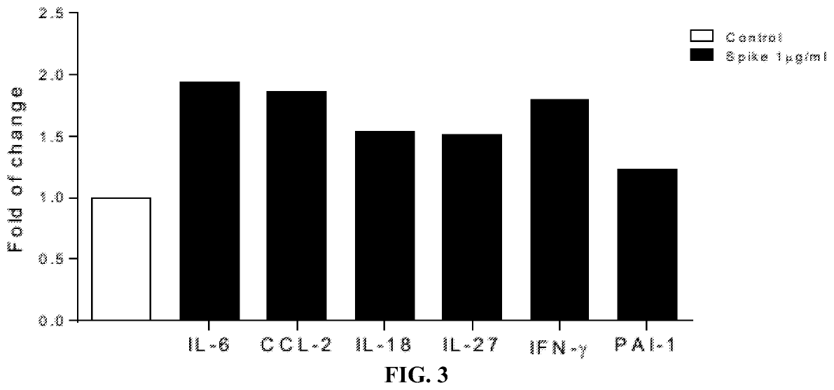

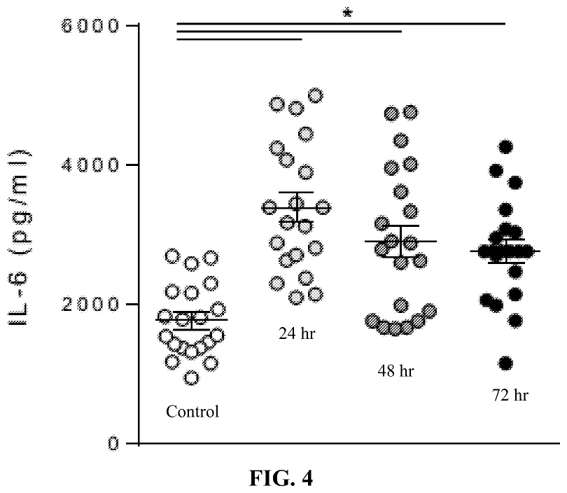

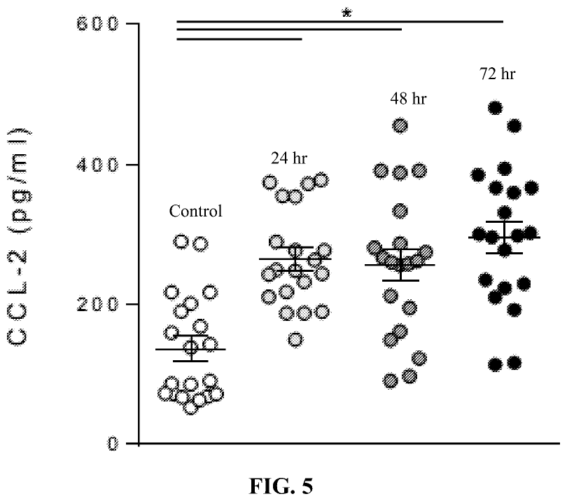

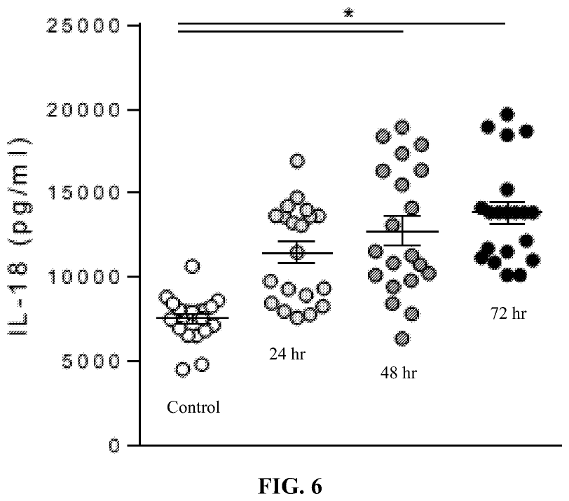

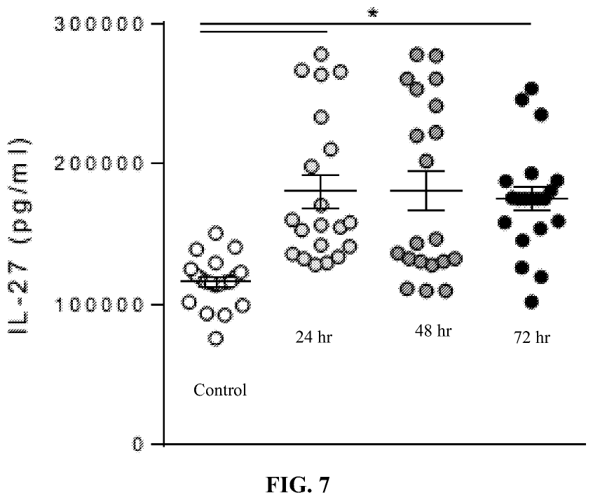

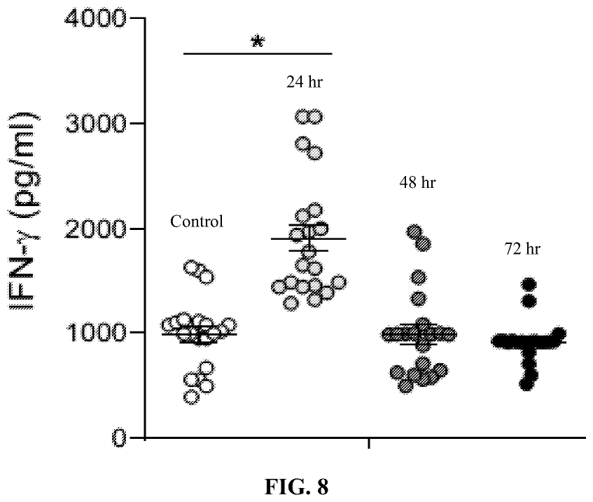

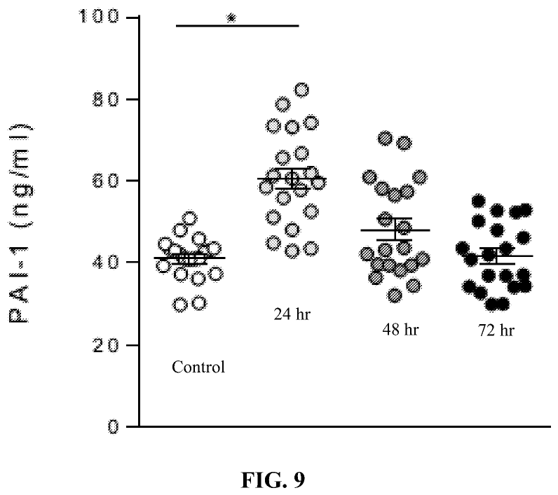

The effects of recombinant SARS-CoV-2 Spike protein on HAECs were analyzed using a cytokine array (FIGS. 2A and 2B) (n=12 supernatants per condition of 3 independent experiments). Each blot represents the immunoreactive staining with each cytokine. The lack of dots represented the negative and blank control. The blots marked inside the box are those selected. The fold of change of cytokines was determined by comparing the pixel intensity of the respective blots to that of the positive control on the same array. The IL-6, CCL-2, IL-18, IL-27, INFγ and PAI-1 cytokines were chosen based on previous studies showing their involvement in COVID-19 infection. The increase of cytokines in recombinant Spike-treated HAECs for 24 hours relative to controls were: 1.94-fold for IL-6; 1.86-fold for CCL-2; 1.54-fold for IL-18; 1.51-fold for IL-27; 1.80-fold for INFγ; and 1.23-fold for PAI-1 (FIG. 3). These results were confirmed in HAECs treated with recombinant SARS-CoV-2 Spike protein for 24, 48 and 72 hours. Treatment with recombinant SARS-CoV-2 Spike protein enhanced IL-6 secretion at 24 (1.92-fold, p<0.0001), 48 (1.64-fold, p=0.0003) and 72 (1.56-fold, p=0.0018) hours (FIG. 4). CCL-2 secretion was increased in a time-dependent manner by recombinant Spike (1.94-fold, p=0.0012 for 24 hours; 1.88-fold, p=0.0018 for 48 hours and 2.17-fold, p<0.0001 for 72 hours) (FIG. 5). IL-18 secretion was time-dependently enhanced by Spike stimulation (1.53-fold, p=0.001 for 24 hours; 1.69-fold, p<0.0001 for 48 hours and 1.84-fold, p<0.0001 for 72 hours) (FIG. 6). Treatment with recombinant Spike protein augmented IL-27 secretion at 24 (1.56-fold, p<0.0001), 48 (1.56-fold, p=0.0003) and 72 (1.51-fold, p<0.0001) hours (FIG. 7). INFγ secretion was only increased after 24 hours of Spike protein treatment (1.92-fold, p=0.0002) (FIG. 8). Finally, treatment with recombinant Spike increased the secretion of the thrombotic marker PAI-1 only at 24 hours (1.37-fold, p<0.0001) (FIG. 9).

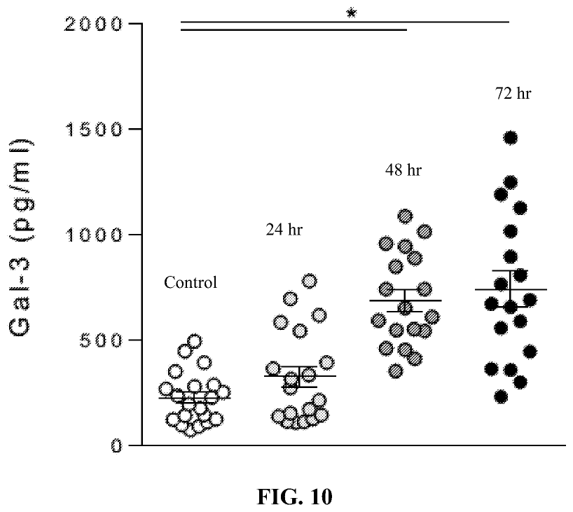

Incubation of HAECs with recombinant Spike significantly induced Gal-3 secretion at 48 (3.15-fold, p<0.0001) and 72 (2.93-fold, p<0.0001) hours (FIG. 10).

### Preventive Gal-3 Pathway Inhibition and SARS-CoV-2 Spike Protein-Mediated Inflammatory Effects in HAECs

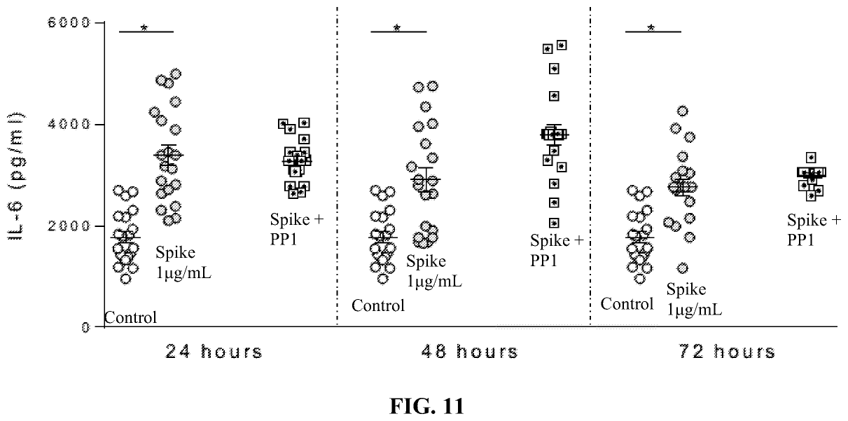

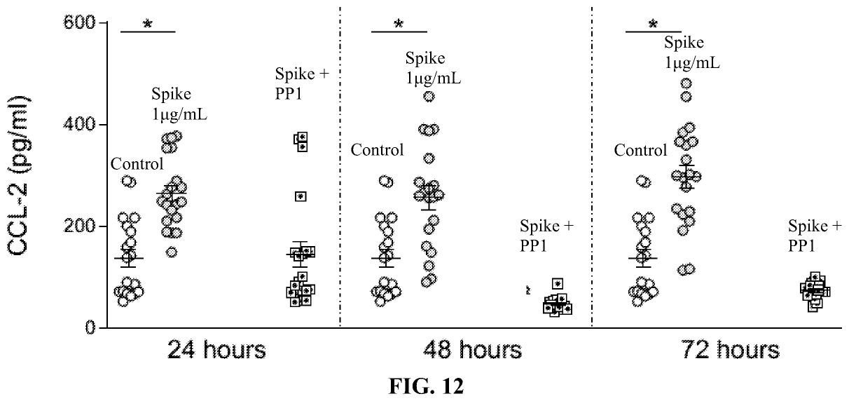

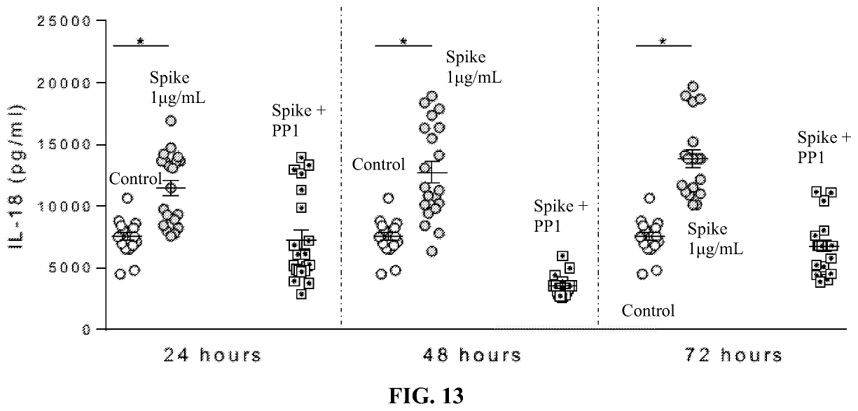

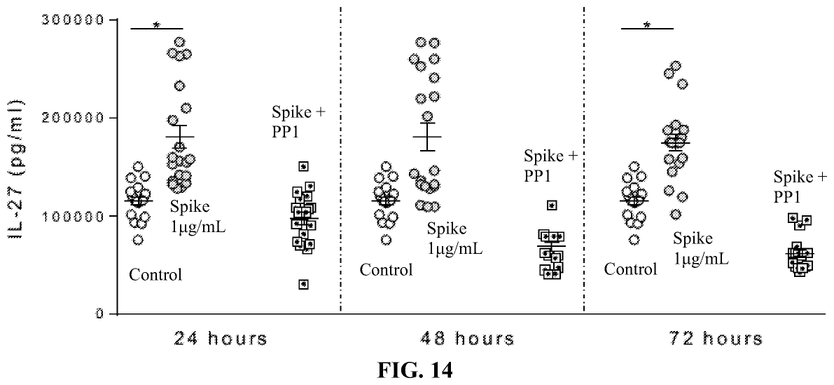

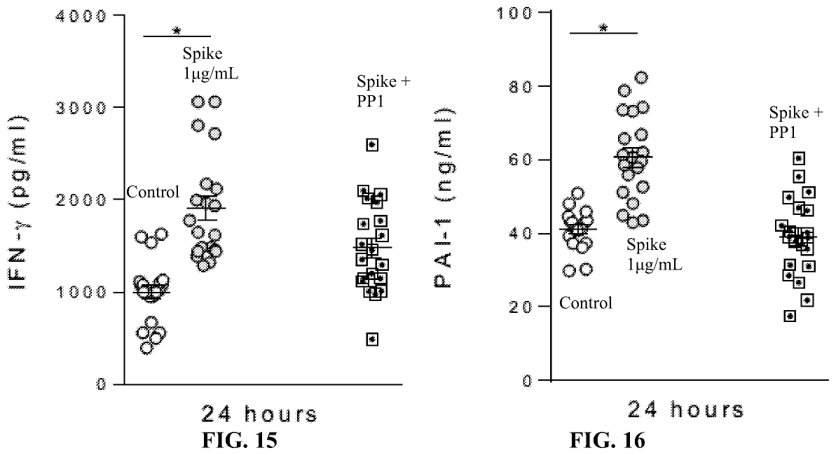

HAECs were pre-incubated with the galectin-3 inhibitor PP1, and then concomitantly treated with SARS-CoV-2 recombinant Spike for 24, 48 and 72 hours. As shown in FIG. 11, PP1 prevented from Spike-induced CCL-2 expression at 24 and 48 hours. PP1 was able to restore normal CCL-2 levels at 72 hours (FIG. 12). The increased expression of IL-18 and IL-27 induced by Spike was prevented by PP1 at all the timepoints (FIGS. 13 and 14).

### Inhibition of the Mineralocorticoid Receptor/Gal-3 Pathway and SARS-CoV-2 Spike Protein-Mediated Inflammatory Effects in HAECs

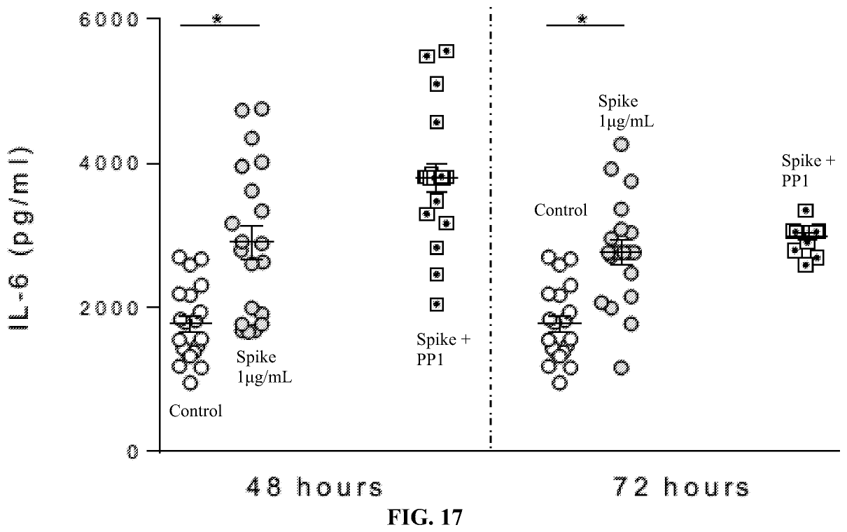

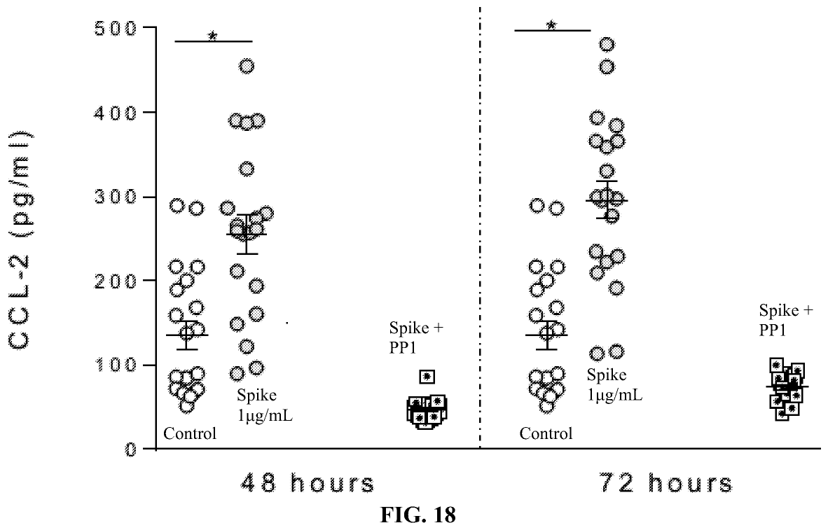

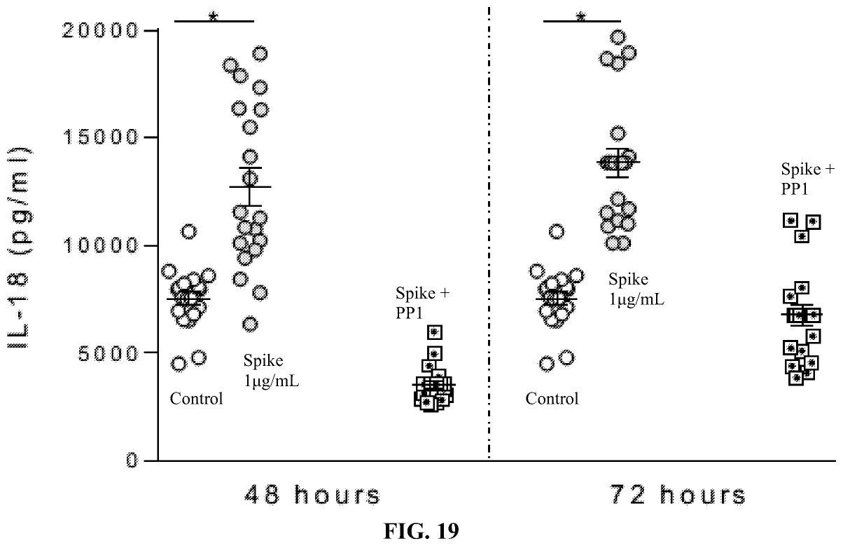

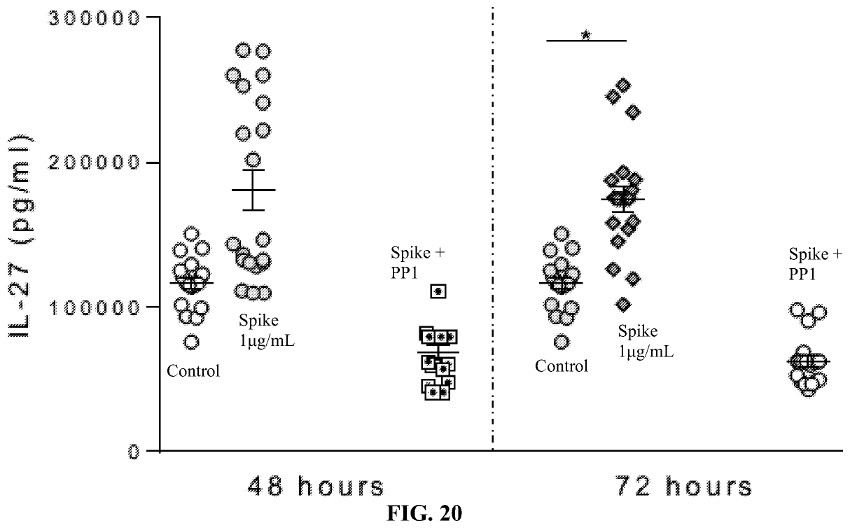

HAECs were pre-treated with recombinant SARS-CoV-2 Spike protein for 24 h to mimic an active SAR-CoV2 infection and then PP1 was added for further 24 h or 48 h. As shown in FIG. 17, PP1 did not block IL-6 increase triggered by pre-treatment with recombinant Spike. CCL-2 upregulation induced by Spike was abolished by PP1 at 48 hours and by PP1 at 72 hours (FIG. 18). The increase in IL-18 and IL-27 induced by pre-treatment with Spike was blocked by PP1 at all the timepoints (FIGS. 19 and 20).

Recombinant SARS-CoV-2 Spike treatment triggered an acute inflammatory response on HAECs as evidenced by a sustained release of cytokines. The Gal-3 inhibitor prevented and reversed recombinant SARS-CoV-2 Spike's most pro-inflammatory effects.

### Discussion

The immunological and physiological functions, and their systemic distribution, makes the endothelial cell a useful target to treat the pathogenesis of COVID-19. In the present study, SARS-CoV-2 Spike induced the secretion of pro-inflammatory cytokines in vascular endothelial cells, reinforcing the idea that vascular endothelial cells are active drivers of COVID-19.

Expression of galectin-3 is upregulated by mineralocorticoid receptor signaling acting as a downstream effector of the Mineralocorticoid receptor (MR) pathway. Indeed, galectin-3 has been described to mediate the effects of MR activation in cardiovascular cells, and has been shown to stimulate inflammation. It is worth noting that single-cell RNA sequencing analysis has identified galectin-3 to be significantly elevated in bronchoalveolar immune cells in patients with severe COVID-19 compared to mild disease. Moreover, a structural homology has been reported between Spike protein and galectin-3. See, for example, Behloul N, Baha S, Shi R, and Meng J. in Virus Res Elsevier B.V.; 2020; 286. Upon ACE2 binding, such ‘Gal-3-like’ domain may be important to stabilization of the viral adhesion and so it may facilitate virus entry. Accordingly, galectin-3 inhibition could disrupt the stability of the SARS-CoV2 binding to the host cell and mitigate the entry of SARS-CoV-2 and the inflammatory response.

Several publications have demonstrated the anti-inflammatory potential of galectin-3 inhibitors. See, for example, Calvier, L. et al. in Arterioscler Thromb Vasc Biol 2013; 33: 67-75 and Martinez-Martinez, E., et al. in Hypertension 2015; 66: 961-969. Galectin-3 inhibition reduces the release of proinflammatory cytokine from immune cells. See, for example, Kei Yip P, Carrillo-Jimenez A, King P, Vilalta A, Nomura K, Cheng Chau C, Michael Scott Egerton A, Liu Z-H, Jayaram Shetty A, Tremoleda J L, Davies M, Deierborg T, Priestley J V, Charles Brown G, Teodora Michael-Titus A, Luis Venero J, Angel Burguillos M. Galectin-3 released in response to traumatic brain injury acts as an alarmin orchestrating brain immune response and promoting neurodegeneration OPEN. Nat Publ Gr 2017 and Ren Z. et al. in Biosci Rep Portland Press Ltd; 2019; 39.

Results herein support that galectin-3 inhibition blocks the proinflammatory response induced by Spike protein and may prevent Spike-induced inflammation, indicating that galectin-3 blockade could exert dual benefits in the treatment of COVID-19. Moreover, the anti-fibrotic effects of galectin-3 inhibitors can have an important role in limiting the development of pulmonary fibrosis, which can be a deleterious consequence in survivors of severe COVID-19.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method of treating fibrosis resulting from a coronavirus infection, comprising administering to a subject in need thereof a therapeutically effective amount of a galectin-3 inhibitor to treat the fibrosis.

2. The method of claim 1, wherein the method achieves at least a 25% reduction in a symptom of fibrosis.

3. The method of claim 1, wherein the method achieves at least a 50% reduction in a symptom of fibrosis.

4. The method of claim 1, wherein the method achieves at least a 90% reduction in a symptom of fibrosis.

5. A method of slowing the progression of fibrosis resulting from a coronavirus infection, comprising administering to a subject in need thereof an effective amount of a galectin-3 inhibitor to slow the progression of the fibrosis.

6. The method of claim 5, wherein the method achieves at least a 25% reduction in the rate of progression of fibrosis compared to the average rate of progression of fibrosis in a subject having been infected by the coronavirus and not having received the galectin-3 inhibitor.

7. The method of claim 5, wherein the method achieves at least a 50% reduction in the rate of progression of fibrosis compared to the average rate of progression of fibrosis in a subject having been infected by the coronavirus and not having received the galectin-3 inhibitor.

8. The method of claim 5, wherein the method achieves at least a 90% reduction in the rate of progression of fibrosis compared to the average rate of progression of fibrosis in a subject having been infected by the coronavirus and not having received the galectin-3 inhibitor.

9. A method of reducing the risk of fibrosis resulting from a coronavirus infection, comprising administering to a subject in need thereof an effective amount of a galectin-3 inhibitor to reduce the risk of fibrosis in the subject.

10. The method of claim 9, wherein the method achieves at least a 25% reduction in the risk of fibrosis.

11. The method of claim 9, wherein the method achieves at least a 50% reduction in the risk of fibrosis.

12. The method of claim 9, wherein the method achieves at least a 90% reduction in the risk of fibrosis.

13. The method of any one of claims 1-12, wherein the fibrosis comprises fibrosis in the subject's lung.

14. The method of any one of claims 1-13, wherein the fibrosis comprises fibrosis in the subject's kidney.

15. The method of any one of claims 1-14, wherein the fibrosis comprises fibrosis in the subject's heart.

16. A method of preventing the development of fibrotic tissue in a subject, comprising administering to a subject in need thereof an effective amount of a galectin-3 inhibitor to prevent the development of fibrotic tissue in the subject, wherein the fibrotic tissue results from a coronavirus infection.

17. A method of treating or preventing a symptom of a coronavirus infection in a subject, comprising administering to a subject in need thereof an effective amount of a galectin-3 inhibitor to treat or prevent a symptom of the coronavirus infection.

18. A method of reducing the impact of a pro-inflammatory cytokine in a patient suffering from a coronavirus infection, comprising administering to a patient in need thereof an effective amount of a galectin-3 inhibitor, in order to reduce the impact of the pro-inflammatory cytokine.

19. The method of claim 18, wherein the pro-inflammatory cytokine is IL-1, IL-2, IL-6, or IL-7.

20. The method of any one of claims 1-19, wherein the coronavirus infection is an infection by SARS-CoV-2.

21. The method of any one of claims 1-20, wherein the galectin-3 inhibitor is a carbohydrate, protein, lipid, nucleic acid, or small organic compound.

22. The method of any one of claims 1-20, wherein the galectin-3 inhibitor is a polysaccharide.

23. The method of any one of claims 1-22, wherein the galectin-3 inhibitor comprises galactose.

24. The method of any one of claims 1-20, wherein the galectin-3 inhibitor is a pectin.

25. The method of any one of claims 1-20, wherein the galectin-3 inhibitor is a modified citrus pectin.

26. The method of any one of claims 1-20, wherein the galectin-3 inhibitor is a pumpkin pectin.

27. The method of any one of claims 1-20, wherein the galectin-3 inhibitor is a pectic compound.

28. The method of any one of claims 1-20, wherein the galectin-3 inhibitor is an antibody.

29. The method of any one of claims 1-28, wherein the subject is an adult human.

30. The method of any one of claims 1-28, wherein the subject is a geriatric adult human.

31. The method of any one of claims 1-30, wherein the galectin-3 inhibitor is administered orally to the subject.

32. The method of any one of claims 1-31, wherein the galectin-3 inhibitor is administered to the subject after the subject has stopped exhibiting any fever, cough, difficulty breathing, fatigue, or digestive system distress due to the coronavirus infection.

33. The method of any one of claims 1-32, wherein the galectin-3 inhibitor is formulated as a component of a nutritional product.

34. The method of any one of claims 1-32, wherein the galectin-3 inhibitor is a component in a food product.

35. The method of any one of claims 1-34, further comprising administering to the subject a therapeutically effect amount of an additional therapeutic agent for treating fibrosis.

36. The method of claim 35, wherein the additional therapeutic agent is a mineralocorticoid receptor antagonist, angiotensin-converting enzyme inhibitor, angiotensin-receptor blocker, anti-inflammatory agent, or combination thereof.

37. The method of claim 35, wherein the additional therapeutic agent is a mineralocorticoid receptor antagonist, angiotensin-converting enzyme inhibitor, angiotensin-receptor blocker, non-steroidal anti-inflammatory drug, inhibitor of IL-6, or combination thereof.
